(19) United States
(12) Patent Application Publication
(10) Pub. No.: US 2026/0365800 A1
(43) Pub. Date: Nov. 19, 2026

(54) METHOD FOR PRODUCING A POLYMER MATRIX LOADED WITH ACTIVE AGENTS BY MEANS OF A GAS, PRODUCT AND ASSOCIATED USE

(71) Applicant: AB7 SANTE, Deyme (FR)

(72) Inventors: Gabriel DALLOUX, Toulouse (FR); Adrien THEZ, Salles sur I'Hers (FR); David N'GUYEN, Toulouse (FR); Arnaud VILBERT, Baziege (FR); Aurélien COURAUD, Toulouse (FR)

(73) Assignee: AB7 SANTE, Deyme (FR)

(21) Appl. No.: 19/133,421

(22) PCT Filed: Nov. 27, 2023
(86) PCT No.: PCT/FR2023/000180

§ 371 Date: May 28, 2025

(30) Foreign Application Priority Data
Nov. 28, 2022 (FR) FR2212441

(51) Int. Cl. C08J 3/20; A61K 47/30

(52) U.S. Cl. CPC C08J 3/20; A61K 47/30; C08J 2300/22

(57) ABSTRACT

Described herein are polymer matrices loaded with active agents. Also described, are methods of obtaining such loaded matrices. More specifically, a method is described for incorporating an active agent into a polymer matrix by means of a gas. The method includes the following consecutive steps: a) impregnating a polymer by means of a gas in the non-supercritical state, and b) incorporating the active agent.

The present invention relates to the field of polymer matrices loaded with active agents and in particular to methods for obtaining said matrices.

More specifically, the invention relates to a method for producing a polymer matrix loaded with active agents by means of a gas, the use of this latter and the product resulting from this method.

The method according to the invention aims to produce matrices loaded with active agents for a variety of applications, in humans or else in the veterinary field where plastics manufacturing processes are often used to produce delivery devices.

These delivery devices often take the form of necklaces or bracelets for a topical application for anti-parasite or insecticide purposes and can also be used to diffuse other active agents such as essential oils.

The plastics manufacturing processes used to create these delivery devices notably include, but are not limited to, the extrusion and injection molding methods.

Extrusion is a continuous transformation process. The polymer is in the form of a granule or powder and is fed into a heated barrel equipped with an endless screw in the case of a single-screw extruder or with two counter-rotating or co-rotating screws in the case of a twin-screw extruder. The material is conveyed, homogenized and plasticized and passes through a die of the desired shape.

Generally, injection molding is a manufacturing method wherein molten material is injected into a pre-prepared mold, then left to cool. As it cools, the material solidifies and then takes on the shape of the mold, before being ejected.

Typically, polymers can be divided into two categories. A distinction is made between thermoplastics and thermosets.

Thermoplastics comprise linear macromolecular chains consisting of monomers, which can induce a reversible physical state based on the heating or the cooling of the material.

They come in two forms: an amorphous form or a semi-crystalline form. The amorphous form is characterized by the glass transition temperature (Tg): when the temperature of the thermoplastic polymer is below this temperature, then this polymer becomes more brittle, and is then said to be glassy. Conversely, when the temperature of the thermoplastic polymer is above the Tg, the macromolecular chains are more mobile and the material is in a rubbery state.

As for the semi-crystalline form, when such a polymer is heated, its structure is disorganized and the material becomes fluid. This phenomenon is called melting and the melting temperature (Tf) is associated with this state. For semi-crystalline polymers, crystalline regions coexist with amorphous regions.

Thermosets, on the other hand, are characterized by a three-dimensional network. These include polycondensed, polymerized or else crosslinked macromolecular units so that when the material is in its final form, said material is in an irreversible state.

For the purposes of the invention, the polymers of interest are selected from the thermoplastic polymers.

For the purposes of the invention, the term “matrix” should be understood to be an element capable of receiving or being impregnated with a substance and of diffusing said substance over time. For the purposes of the invention, the polymer matrix thereby acts as a reservoir for the active agent enabling it to be released over a prolonged period.

An active agent reservoir is understood to be the polymer matrix loaded with active agent, wherein the active agent is homogeneously distributed during the incorporation method. It must thus be understood that the invention does not comprise compartmentalization of the active agent within the matrix.

For the purposes of this invention, the term “incorporation” can be defined as the act of incorporating or introducing a liquid or solid active agent into a polymer matrix as defined hereinbefore.

The terms diffusion, migration or release can also be used interchangeably to relate to the exit of the active agent from the polymer matrix, from the inside to the outside thereof. In the remainder of the application, the terms matrix, polymeric matrix, polymer matrix, solid matrix, thermoplastic matrix or else polymer will be used interchangeably to define the polymer matrix according to the invention.

### STATE OF THE ART

It is known from the background art FR2901172, which discloses a method for incorporating active agents into a polymer matrix. This method addresses the problem of incorporating heat-sensitive active agents into a polymer matrix. To this end, a method is proposed consisting of heating polymers from 1 °C to 5 °C above their glass transition temperature in order to incorporate a composition of heat-sensitive active agents at room temperature. The polymers disclosed are ethylene-vinyl acetate copolymers (EVA) or polyether block amides (PEBA), and these latter are described as having glass transition temperatures (Tg) between 20 °C and 80 °C. However, this method does not make it possible to obtain incorporation conditions of the level of the method according to the invention. Test number 5 of Example 2 (FIG. 1) shows, inter alia, the comparative results of incorporating active agents between a PEBA and EVA matrix, without CO2 impregnation as described in the background art and with CO2 incorporation using the method described according to the present invention. The results show a superiority of the method according to the invention. Indeed, EVA and PEBA type polymers have very low and negative Tg, and it is not possible to incorporate active agents effectively in the proportions indicated, by repeating the steps of the method as described in the background art. It may also be added that this method of the background art is not easily extended to other matrix-active agent pairs, i.e. the reproducibility of this incorporation method on other polymers or with other active agents is not guaranteed. Moreover, the use of PEBA is not a feasible solution from an industrial viewpoint, as this type of polymer confers too much flexibility on the resulting device, making it incompatible with the majority of intended uses and its cost is extremely high. There are also other problems inherent in the use of these polymer matrices. One example in this regard is the efficacy of the product over time. Indeed, it is necessary to have a stable product with prolonged efficacy, which does not lose its incorporation ability over time and which can release larger quantities of active agents over a longer period of use. Finally, FR2901172 does not disclose any method involving a gas, nor does it suggest the use thereof to aid the incorporation of active agents.

Numerous background art documents describe the use of a fluid to impregnate a polymer but the temperature and pressure conditions are those of a supercritical state, therefore under very high pressure and/or temperature conditions. U.S. Pat. No. 4,598,006 discloses a method for impregnating thermoplastic polymers using a gas as a volatile blowing agent. However the method consists in a first step of dissolving an active agent in said volatile blowing agent under supercritical or near-supercritical conditions. The thermoplastic of interest is then brought into contact with the volatile blowing agent containing the active agent to be incorporated at very high pressures (2500 psi or 173 bar). The volatile blowing agent is used both to dissolve the active agent and to swell the thermoplastic for incorporation, which is only carried out under supercritical conditions or close to the supercritical state. Furthermore, this document only discloses a method in which the incorporation into the polymer is carried out simultaneously with its impregnation and under very high pressure conditions, which are therefore complex to implement, as they require special equipment to generate and withstand such pressures. Moreover, as shown in the examples, the incorporation rates with this type of method remain very low, with less than 6.5% active agent in the finished product resulting from this method using supercritical CO2 or close to a supercritical state. This method does not offer any solutions for improving the incorporation of active agents using a gas under non-supercritical pressure conditions.

Regarding the use of gas on polymers, the background art contains numerous documents on techniques for foaming or expanding thermoplastics with the aim of making the final material lighter. But these documents do not relate to the field of the present invention nor the issues of incorporating active agents related thereto.

Indeed, Duarte et al. mention in two publications (“Subcritical carbon dioxide foaming of polycaprolactone for bone tissue regeneration”, The Journal of Supercritical Fluids, Volume 140, October 2018, Pages 1-10; and “PDLLA enriched with ulvan particles as a novel 3D porous scaffold targeted for bone engineering”, The Journal of Supercritical Fluids, Volume 65, May 2012, Pages 32-38), the use of a method using CO2 incorporation but in fields far removed from the present invention and for very different purposes. Indeed, Duarte et al. seek to produce 3D polycaprolactone or PDLLA/Ulvan frameworks/support structures in the field of in situ bone regeneration. Their aim is to be able to use a single-step method using carbon dioxide to create a 3D polymer structure in situ, by injecting said polymer in the form of an expanded foam. Their aim is to obtain a highly porous structure within which they can incorporate an active agent. Their method leads to the desired structure using a pressure of 50 bar and a temperature for incorporating the gas of at least 45 degrees. Unlike this background art and all those which define porous structures obtained by impregnating by means of a gas, the method of the present invention does not lead to obtaining an expanded foam or any other porous structure. Moreover, the method according to the present invention carries out the impregnation of the gas into the polymer at a temperature strictly less than 40 degrees therefore much less than the temperature of the background art, and at a pressure of less than 35 bar. Moreover, the 50 bar pressure used according to the background art requires sophisticated equipment and more stringent safety conditions than in the case of the pressures used according to the present invention. Tests described in Example 12 show scanning microscopy analyses on the polymer matrix impregnated according to the invention and no porosity is observed. Nothing in the background art documents suggests the method according to the invention, nor does it disclose the matrix obtained by the method according to the present invention.

Powel et al. in her publication (Chemotherapeutic implants via subcritical CO2 modification, Biomaterials. 2007 December; 28(36):5562-9) mentions a method for incorporating a chemotherapy treatment into a PLA resorbable polymer using CO2 compressed to 45 bar at a temperature of 40 °C. The step of impregnating the polymer with CO2 requires heating to 40 °C, unlike the method according to the invention, which is preferentially carried out at 25 °C Furthermore, the active agent loaded into the polymer is rapidly released within 2 days. This document does not disclose a method for improving the incorporation of an active agent into a polymer by means of a gas, the impregnation of which preferentially takes place at 25 °C and at a pressure of less than 35 bar, and the release of which extends over as long a period as possible.

US20210269607 discloses a method for incorporating active agents into a polymer, limited to polybutylene succinate (PBS), said method having to provide a structure with pores or cavities in order to achieve the incorporation. Said porous structure is obtained on the one hand by the mandatory presence of an additive modifying the structure of the polymer, and on the other hand by a gas impregnation step carried out with CO2 under supercritical conditions (100 bar/40 ° C). US20210269607 therefore does not disclose the use of a method at non-supercritical temperatures or pressures, nor does it suggest the possibility of incorporating active agent without requiring a structure with pores or cavities.

WO2014146864 discloses a method consisting of mixing a starch-based product with a phenolic derivative, a solvent and a buffer and exposing said mixture to a fluid at a sufficiently high but “sub-critical” temperature and pressure to cause a chemical reaction and lead to a modified starch-based product. The fluids used are limited to liquids such as water or any other aqueous ionic liquid. Furthermore, the temperature at which the method is carried out is 100 °C. WO 2014146864 in no way discloses the incorporation of a gas into a polymer, the aim of which is to improve the incorporation of an active agent.

### PRESENTATION OF THE INVENTION

There is therefore a need to provide a method, achievable at temperatures and pressures that do not correspond to supercritical conditions, for obtaining an increase in the amount of active agent that can be incorporated into a polymer matrix in a stable and prolonged manner which can overcome the disadvantages mentioned, regardless of the nature of the matrix or active agents employed. In some cases, there is also a need to speed up the method for incorporating active agents into matrices. Furthermore, in some cases, the incorporation of active agents proves impossible, either because of the nature of the polymer matrix, or because of the nature of the active agent. There is therefore also a need to make said incorporation achievable.

The terms maximum incorporability, maximum incorporation, maximum incorporation rate, increased amount of active agent incorporable into the matrix, increased amount of incorporated active agent can be defined as being equivalent and relating to the ability of a matrix according to the invention to incorporate a certain proportion of active compounds. This includes a notion of an achievable maximum.

One aim of the invention is thus to provide a method for incorporating an active agent into a polymer matrix by means of a gas which is not in a supercritical fluid state enabling the maximum incorporation of a polymer to be improved or the incorporation method to be accelerated or the incorporation to be made possible. The objective of the invention is also the product obtained by this method and the use of a gas to improve the incorporation rate of a polymer matrix. The supercritical conditions of a fluid or a gas are well known to the skilled person and are defined by precise pairs of temperature and pressure. They correspond, notably for carbon dioxide, to the precise temperature/pressure pair of 31 °C/73 bar, i.e. very high pressure conditions. For nitrogen, the supercritical conditions correspond to a precise temperature/pressure pair of −147 °C/34 bar, i.e. extreme temperatures. A gas in the non-supercritical or “subcritical” state, for use according to the invention, is therefore understood to be a gas whose temperature and pressure value pairs used are lower, below the thresholds of said value pairs known to the skilled person defining their critical point, in terms of temperature and/or pressure.

Moreover, the use of a gas in the non-supercritical state is a significant advantage in terms of the ease with which the method can be carried out. Indeed, the industry remains reluctant to use very high-pressure methods such as those used in technologies using gas under supercritical conditions. These methods require sophisticated and often expensive equipment. Moreover, these very high pressures remain delicate parameters in the consideration of the safety of users.

The applicant has been able to develop a method for impregnating a gas, which is not in a supercritical fluid state, giving the impregnated matrix improved properties for incorporating active agents and for maintaining these capabilities over time. More particularly, the applicant proposes a new method for incorporating active agents in two successive steps: a step of impregnating the polymer matrix by means of a gas that is not in a supercritical fluid state followed by a step of incorporating the active agent into the impregnated polymer matrix.

The method according to the invention makes it possible, on the one hand, to obtain a polymer matrix which has an improved ability for incorporating active agent, and on the other hand, said polymer matrix retains its incorporation ability even after storage. There is thus an effective and long-lasting increase in the incorporation ability by virtue of the method according to the invention. With the present invention, the applicant is therefore proposing a solution to the problems of improving the maximum incorporation rate of active agents in a polymer matrix, or accelerating their incorporation, or making incorporation possible, and this on several types of polymers by virtue of a prior step of treating the polymer with a gas in the non-supercritical state.

The method for incorporating active agent into a polymer matrix by means of a gas according to the invention comprises the following successive steps:
a) Impregnating a polymer by means of a gas in the non-supercritical state,
b) Incorporating the active agent.

Successive steps according to the invention is understood to mean that the steps of gas impregnation a) and of incorporating the active agent b) are not simultaneous. Incorporation can take place either immediately after the gas impregnation step, after a few hours or after several days, or even weeks after said impregnation step.

Thus the applicant proposes a new method for incorporating at least one active agent into a polymer matrix in two successive steps, comprising a first step of impregnating the matrix with a gas that is not in a supercritical fluid state, followed by a second step of incorporating said active agent into the impregnated matrix. The concept of two successive steps is an important feature of the method according to the invention as it offers several advantages. Indeed, surprisingly, the applicant has shown that impregnation of the gas and incorporation of the active agent, when performed simultaneously, did not result in an incorporation of the active agent into the polymer. Furthermore, surprisingly, the applicant was able to observe that when impregnation was carried out in the absence of active agents, upstream of an incorporation step, this allowed a higher matrix incorporation rate to be obtained than conventional methods. Even more surprisingly, the applicant was also able to demonstrate that the use of an impregnation step with a non-supercritical fluid resulted in a gain in polymer incorporability, and that the polymer retained its greater incorporation ability over time. Indeed, a lasting effect of the modification caused by gas impregnation on the polymer has been observed which enables the active agent incorporation step to be staggered in time from the first step of impregnating the matrix by the gas, without altering the improved incorporation properties. The method thus advantageously enables impregnated matrices to be preserved or stored before being subjected to incorporation at a later date when required.

The present invention therefore relates to a method for incorporating active agent into a polymer matrix by means of a gas which comprises the following successive steps:
a) Impregnating a polymer by means of a gas in the non-supercritical state,
b) Incorporating the active agent,
   whose first impregnating step gives the impregnated polymer matrix improved properties for incorporating active agents and for maintaining these abilities over time.

In the remainder of the present description, “impregnation” refers to the step of impregnating a polymer by means of a fluid, preferentially a gas, in the non-supercritical state; “incorporation” refers to the step of incorporating an active agent into the polymer matrix. In the remainder of the present description, it will be understood that the term “polymer” refers to the polymers of interest cited previously, namely thermoplastics generally.

The polymers constituting the solid matrix are selected notably from non-biodegradable thermoplastic polymers, selected from the group formed by:
Polyolefins and derivatives thereof selected from polyethylenes (PE), polypropylenes (PP), ethylene-vinyl acetate copolymers (EVA) and ethylene butyl acrylates,
Polyamides, copolyamides and derivatives thereof.
Other vinyl polymers such as polyvinyl chloride (PVC) or styrenics and derivatives thereof selected from polystyrene-block-poly(ethylene-ran-butylene)-block-polystyrene copolymers (SEBS), polystyrene-polyisoprene-polystyrene copolymers (SIS), polystyrene-polybutadiene-polystyrene copolymers (SBS).
Other thermoplastic elastomers such as thermoplastic polyurethane (TPU), polyether block amides (PEBA), ethylene-propylene-diene monomer (EPDM) or derivatives thereof (PP/EPDM).
Polyesters such as polyethylene terephthalate (PET), polybutylene terephthalate (PBT) or polytrimethylene terephthalate (PTT).

Alternatively, the polymer constituting the solid matrix is a biosourced and/or biodegradable thermoplastic polymer which can be:
a polyester or copolyester, selected from polycaprolactones, polylactides (PLA), polyesteramides, aliphatic and aromatic copolyesters,
an agro-polymer selected from polysaccharides, starch and derivatives thereof, cellulose compounds and derivatives thereof, milk protein derivatives or a mixture of all these polymers.
polymers derived from microbial synthesis such as polyhydroxyalkanoates (PHA), poly(β-hydroxybutyrate) (PHB) or all derivatives thereof.

In a preferred alternative, polymers suitable for use as a matrix in the invention are preferentially selected from ethylene vinyl acetate (EVA), polyamides, polyether block amides (PEBA), polypropylenes (PP), polyethylenes (PE), thermoplastic polyurethanes (TPU), polyvinyl chlorides (PVC), polystyrenes such as polystyrene-block-poly(ethylene-ran-butylene)-block-polystyrene (SEBS) or polystyrene-polyisoprene-polystyrene (SIS), polyesters, polylactic acids (PLA) or biodegradable polymers, agro-polymers, bioplastics or biosourced thermoplastics, biopolyesters and derivatives thereof.

The active agents used in the invention are active agents in powder or liquid form. Preferentially, the active agents are lipophilic in nature.

By lipophilic active agents, we mean that the active agents may have been previously solubilized or dispersed in a fatty or oily phase, or else that they have an affinity for the fatty or oily phases.

The active agents suitable for incorporation into the polymer matrix according to the method of the invention can be selected from active agents known to the skilled person such as chemical pharmaceutical active agents or active agents of natural origin, essential oils, or mixtures thereof. The active agents for use according to the invention can have different indications notably in the veterinary field, such as insecticides or repellents, anti-parasitics, painkillers, sedatives or anti-stress agents.

The active pharmaceutical agents may be selected from the active agents known to the skilled person for use in the veterinary field.

Insecticidal or repellent active pharmaceutical agents may be selected from plant pyrethrum, pyrethroids, pyrethrins and derivatives thereof, carbamates, formamidines, carboxylic esters, N,N-diethyl-3-methylbenzamide (DEET), icaridin, phenylpyrazoles, organophosphorus compounds, organohalogen compounds, neonicotinoids, avermectins and derivatives thereof, spinosyns, nootkatone and derivatives thereof, isoxazolines or mixtures thereof.

Antiparasitics can be selected from anthelmintics, endectocides, isoxazolines or mixtures thereof. Although they have a systemic action against parasites, some endectocides can also have an insecticidal effect.

Painkillers or sedatives can be selected from active agents known to the skilled person for these activities, and can be notably selected from cannabinoid derivatives devoid of tetrahydrocannabinol (THC), such as for example THC-free cannabidiol (CBD) or THC-free cannabigerol (CBG) or mixtures thereof.

Anti-stress agents can be selected from the cannabinoid derivatives cited previously, but also from pheromones or essential oils.

Essential oils, for use as active agents according to the present invention, can present multiple indications and properties.

An essential oil refers to a concentrated plant extract of volatile aromatic compounds such as, but not limited to, geraniol, limonene, menthol, linalool, citriodiol, citronellal and lactones, without prejudice to the presence of other constituents of essential oils or mixtures thereof.

The essential oil is preferentially selected from the essential oil of lavandin, lavender, orange, margosa, eucalyptus, citronella, lavender, neem, peppermint, spearmint, pennyroyal, corn mint, wintergreen, basil, rosemary, cedarwood, citronella, clove, geranium, thyme or a mixture thereof.

For the purposes of the present application, it is understood that the active agents cited may be mixed in particular formulations and that the applicant does not intend to limit itself to just one of these categories. It is also understood that the active agent used can also be a mixture of active agents and notably a mixture of the active agents previously mentioned.

For the purposes of the present application, it is understood that the active agents may also be additive-type ingredients which the skilled person wishes to introduce into the polymer matrix for their activity within the composition. The applicant has demonstrated that the method according to the invention has the advantage of enabling these ingredients to be incorporated more easily or in greater proportions. Thus, by way of non-limiting example of an additive that can be incorporated as an active agent according to the invention, it is possible to cite a plasticizer, perfume, perfume carrier, preservative, colorant, anti-oxidant or UV stabilizer. As a preferential example, the applicant has demonstrated that the incorporation of a perfume carrier on a polymer matrix is improved using the method according to the present invention. In a particular case according to the invention, the perfume carrier used is monopropylene glycol.

In one embodiment, the active agent comprises at least one essential oil.

In another embodiment, the active agent also comprises an auxiliary oil. By auxiliary oil, we mean for example a vegetable oil which can preferentially but not exclusively be selected from almond oil, linseed oil, rapeseed oil, macadamia oil or else hemp oil, or THC-free CBD oil.

In one alternative embodiment, the active agent is a mixture comprising at least an essential oil, an auxiliary oil, optionally with an active agent of plant or chemical origin and any other additional active agent desired to impart the desired properties to the polymer matrix.

### Impregnation Step

The first step of the method according to the invention is thus that of impregnating the polymer matrix with a non-supercritical fluid.

Impregnation can be defined as the act of subjecting a polymer to the pressure of a fluid, under specific pressure and temperature conditions, these conditions enabling it to enter the polymer network.

In one embodiment, the non-supercritical fluid is a gas in the non-supercritical state, such as compressed air, carbon dioxide (CO2) or dinitrogen (N2), preferentially carbon dioxide (CO₂) or dinitrogen (N₂) in the non-supercritical state. Indeed, the applicant does not exploit the ability of these gases in the supercritical state, namely their solvent property, since this impregnation step according to the present invention does not serve to solubilize the active agent. Moreover, this solvent effect of the supercritical fluid on the active agent would only be obtained in a method where impregnation would be simultaneous with incorporation, and yet the applicant has found that impregnation simultaneous with incorporation of liquid active agents into the matrix fails to solve the problem posed in the present invention. In the method according to the invention, improved incorporation of the active agent into the matrix requires impregnation to be carried out in a step upstream of incorporation, and at temperature and pressure values of the gas below those known for the supercritical state.

During this impregnation step, the polymers are placed in a hermetically-sealed pressure-resistant reactor fitted with an opening flange. This opening flange is itself connected to a gas inlet valve for managing the entry of gas into the reactor. The whole assembly is then connected to a gas source and comprises means for controlling pressure, temperature and evaluating other necessary physico-chemical variables known to the skilled person.

The polymers used during the impregnation step in the invention are in the form of granules, beads, powders or any other form known to the skilled person suitable for stirring in an incorporation tank. Impregnation according to the method of the invention is not carried out on molded or already formed parts. In this respect, it is also important to exclude any form of blowing of the material with the gas from the impregnation step; there is therefore no volatile blowing agent and the invention is not intended to cause the matrix to foam.

Once the polymer has been placed in the reactor, the reactor is then pressurized. The impregnation step thus takes place in the presence of a pressurized gas.

The applicant was able to determine that the improvement in the matrix incorporation rate and the changes brought about by the non-supercritical fluid were obtained from a gas pressure used during the impregnation step, equivalent to 5 bar.

A pressure range in which the gas increases the maximum incorporation into the matrix has thus been evaluated. Unexpectedly, the applicant was able to demonstrate that when performing gas impregnation over a pressure range of between 5 and 50 bar, preferentially between 5 and 35 bar, the maximum incorporation of the polymer increased based on the pressure. The applicant has determined that above 35 bar, the effect on incorporation seems to reach a plateau and that between 1 bar (atmospheric pressure) and 5 bar, changes in incorporation are not significant.

In one embodiment, the pressure ranges likely to be used during the impregnation step of the method can be between 5 and 35 bar, preferentially between 20 and 35 bar, more preferentially at the pressure of 30 bar.

Another important factor in the impregnation step to be combined with pressure is that of temperature. Indeed, the applicant has shown that the maximum incorporation obtained for the same matrix at the same pressure can vary based on the gassing temperature. This gassing temperature can be defined as the temperature at which the matrix is placed in the presence of gas in the reactor. It is also understood that the gassing or pressurizing temperatures are defined as the temperature applied to the entire system formed by the polymer matrix and the gas within the reactor.

In the context of the polymers and thermoplastics of the invention, one might think that the higher the temperature, the more the polymer network would tend to relax and to leave more spaces or cavitations. The spaces thus created could then enable better incorporation following the impregnation step.

And yet, surprisingly, in the method according to the invention, the applicant was able to demonstrate that the pressurization temperatures had to be relatively low temperatures for a polymer. Indeed, the temperatures likely to be employed during the impregnation step of the method are strictly less than 50 °C, preferentially strictly less than 40 °C, preferentially between 15 and 35 °C, more preferentially between 20 °C and 30 °C, with the best results being obtained at 25 °C. As a result, surprisingly, the effect of the pressurization temperature on the maximum incorporation rate of the matrix is all the greater the lower said temperature.

With regard to impregnation time, notwithstanding the time required for the system to build up pressure, this is an inverse function of the pressure applied. Indeed, the higher the pressure applied to the system, the shorter the impregnation time of the polymer with the non-supercritical fluid and vice versa.

For a pressure range between 5 and 35 bar, the impregnation time can be between 150 min and 5 min respectively.

### Incorporation Step

The second step of the method is a step of incorporating the polymer matrix. Impregnation can then be considered as a step of pre-treating the matrix prior to the incorporation thereof.

During the incorporation step, the impregnated matrix is placed in a stirred tank equipped with a heating means known to the skilled person. Surprisingly, the applicant found that the incorporation rate of the previously impregnated matrix was always higher than that of the unimpregnated matrix, irrespective of the incorporation temperature.

At the end of this incorporation step, the matrix can be defined as an incorporated matrix, or loaded with active agents, ready for shaping according to known plastics manufacturing techniques.

The incorporation temperatures depend on the nature of the polymers, and more particularly on the active agent-matrix pairs, but for the polymers of the invention, it is advisable to work at an incorporation temperature greater than the Tg but nevertheless less than the melting temperature of the polymer. The skilled person will know how to adapt the incorporation temperature to the nature of the polymer matrix and the active agent(s) selected. Taking these parameters into account, the best results for incorporating active agents into the polymers tested according to the invention were obtained for an incorporation temperature ranging from 30 °C to 100 °C, preferentially from 35 °C to 65 °C.

The result is thus a transition state at the molecular level where the amorphous part of the polymers is fluidized, i.e. the chains making up the polymer network are more mobile when this temperature is reached.

In these temperature ranges, the use of the pre-impregnation step of the matrix by means of gas in the non-supercritical state of the invention improves the maximum incorporation of the matrix independently of the incorporation temperature. It is therefore possible to reduce the incorporation temperature for a given and known incorporation rate using the method according to the invention. On an industrial scale, such a method would then be less energy-intensive by virtue of the matrices impregnated according to the method of the invention.

In addition to the effects improving the maximum incorporation of a given polymer, the applicant was able to demonstrate that impregnation also made it possible to reduce the duration of the incorporation step with respect to a conventionally used loading method and that this effect was all the more noticeable for EVA. Thus, the method used is faster, therefore less expensive and more efficient for incorporating the active agents.

On the other hand, for a similar incorporation time for a given polymer, the applicant's method always shows an increase in the maximum incorporation rate with respect to a conventional method even on matrices described as difficult to incorporate, or with active agents known to be difficult to incorporate.

Without being exhaustive, these difficult matrices may for example belong to the class of polypropylenes (PP) or polyethylenes (PE).

The applicant has thus been able to develop a method for producing a polymer matrix loaded with active agent, said method comprising an impregnation step prior to a step of incorporating said active agent. Said loaded matrix, obtained at the end of the method, thus benefits from a better incorporation rate of the active agent with respect to the methods conventionally used in this field. By better incorporation rate, we mean an incorporation rate of active agents which is increased by at least 15% and up to 100% depending on the nature of the polymers and active agents.

At the end of the method according to the invention, the loaded matrix can be subjected to shaping by means known to the skilled person in order to form an active object of the desired shape ready for use. Thus, the polymer matrix can be obtained in the form of granules, said granules can then be injected on a press or other injection or extrusion means in order to give the matrix the desired shape. In a non-limiting way, the devices resulting from the loaded matrix obtained by the method according to the invention can be used in the form of necklaces, bracelets, harnesses, ear tags or any other form of active agent delivery device, preferentially for animals. To produce these delivery devices, such as necklaces for example, it is necessary to obtain a homogeneous polymer matrix in order to ensure optimum diffusion and good distribution of the active agent therewithin. The method according to the invention obtains such a matrix which is homogeneously incorporated with at least one active agent.

The present invention therefore relates to a method for producing a polymer matrix loaded with at least one lipophilic liquid active agent using a gas characterized in that it comprises the following successive steps:
a) Impregnating the polymer with said gas in the non-supercritical state at a temperature strictly less than 40 °C and at a pressure of between 5 and 35 bar;
b) Incorporating the active agent at a temperature of between 30 °C and 100 °C.

The invention also relates to the method characterized in that the non-supercritical gas of the impregnation step is selected from carbon dioxide (CO2) or dinitrogen (N2) and in that the gas impregnation step is preferentially carried out at a temperature of between 15 °C and 35 °C, preferentially between 20 °C and 30 °C, preferentially for a time of between 5 min and 150 min.

The invention therefore relates to the method described previously characterized in that the polymer is a thermoplastic, preferentially selected from ethylene vinyl acetate (EVA), polyamides, polyether block amides (PEBA), polypropylenes (PP), polyethylenes (PE), thermoplastic polyurethanes (TPU), polyvinyl chlorides (PVC), polystyrenes such as polystyrene-block-poly(ethylene-ran-butylene)-block-polystyrene (SEBS) or polystyrene-block-polyisoprene-block-polystyrene (SIS), polyesters, polylactic acids (PLA) or else biodegradable polymers, agro-polymers, bioplastics or biosourced thermoplastics, biopolyesters.

The method according to the invention is characterized in that the active agent incorporated into the polymer matrix is selected from chemical pharmaceutical active agents, active agents of natural origin, essential oils, vegetable oils or mixtures thereof.

In another embodiment, the invention also relates to the polymer matrix loaded with at least one lipophilic liquid active obtained according to the method according to the invention, characterized in that the incorporation rate of said active agent is increased by at least 15% compared with the incorporation of said active agent carried out in said matrix according to a method without a gas impregnation phase.

The present invention also relates to the use of a gas in the non-supercritical state to impregnate a polymer matrix characterized in that said gas is impregnated at a temperature strictly less than 40 °C and at a pressure of between 5 and 35 bar, to increase the amount of active agent incorporated into said polymer matrix. Preferentially the gas is selected from carbon dioxide (CO2) or dinitrogen (N2).

The method of the invention will be better understood through the following non-limiting examples and figures.

The examples described below confirm the advantage of the method according to the invention over a range of different polymers and active agents.

The polymers tested notably included non-biodegradable polymers such as EVA, polyamide, polyethylene, polypropylene, polyester or biodegradable polymers such as biopolyester or casein-derived polymers.

Various types of active agents were also tested, notably:
chemical insecticides or repellents, such as pyrethroids, pyrethrins and derivatives thereof, organophosphorus compounds, notably icaridin, permethrin, phenotrin and diazinon,
insecticidal compounds of plant origin, such as plant pyrethrum,
vegetable oils, known to be difficult to incorporate such as Neem oil and sweet almond oil,
essential oils such as lavandin oil, peppermint oil or cedarwood oil,
mixtures composed of essential oils, auxiliary oils and/or plant or chemical active agents, such as mixtures of linseed oil, plant pyrethrum and lavandin essential oil (referred to as Active Agent A in the examples below), or mixtures of sweet almond oil, peppermint essential oil and cedar essential oil (referred to as Active Agent B in the examples below) or a mixture of lavandin, geraniol and pyrethrum (referred to as Active Agent C in the examples below) or a mixture of icaridin, lavandin essential oil, geraniol and coconut oil (referred to as Active Agent D in the examples below)
additives such as perfume carriers, like monopropylene glycol (referred to as Active Agent E in the examples below).

### FIGURES

FIG. 1 shows the results of comparative tests of the method according to the invention with a conventional incorporation method for several active agent-matrix pairs, as described in Example 2.

FIG. 2 shows the test results for the maximum incorporable active agent based on the impregnation pressure, as described in Example 3

FIG. 3 shows the comparative test between the method of the invention and a conventional method for active agents considered difficult to incorporate, as described in Example 4.

FIG. 4 shows how the incorporation rate of the matrix is maintained over time after impregnation as described in Example 5.

FIG. 5 shows a study of the influence of CO2 on maximum incorporation based on the incorporation temperature, as described in Example 6.

FIG. 6 shows the maximum incorporation obtained with a biopolyester matrix, with different active agents, with or without CO2 treatment, as described in Example 7.

FIG. 7 shows the maximum incorporation obtained with different insecticide active agents for different polymer matrices, with or without CO2 treatment, as described in Example 8.

FIG. 8 shows the cumulative amount of active agent A released over time by an EVA matrix obtained by a method with or without CO2 treatment, as described in Example 9.

FIG. 9 shows the cumulative amount of active agent B released over time by a polyamide matrix obtained by a method with or without CO2 treatment, as described in Example 10.

FIG. 10 shows the scanning microscope image of the polymer matrix obtained according to the method of the invention as described in Example 12.

FIG. 11 shows the scanning microscope image of the polymer matrix without gas impregnation as described in Example 12.

FIG. 12 shows the maximum incorporable for an essential oil only on an EVA matrix with or without CO2 treatment as described in Example 13.

### EXAMPLE 1

### Conduct of the Method According to the Invention on an EVA Polymer Matrix

### Matrix Impregnation:

EVA granules devoid of active agents are placed in an impregnation reactor fed by a CO₂ source. In order to maintain this gas in the non-supercritical state, the temperature is set at 25 °C and the pressure applied to the granules is 30 bar. The granules are then maintained at these conditions for a duration of 120 min, before being subjected to the incorporation step.

### Matrix Incorporation:

The now impregnated EVA granules are then incorporated with the active agent.

To achieve this, the granules are placed in an incorporation reactor in the presence of the active agent at a fixed temperature of 61 °C. This reactor is subjected to stirring for an incorporation time of 2 h 30 min at the end of which granules loaded with active agent are obtained. At the end of this step, said granules constitute the loaded or incorporated matrix.

Said granules can then be subjected to injection on a press or other means known to the skilled person in order to form an active object of the desired shape ready for use. Kinetic and assay studies on a standardized shape can then be used to compare the effects of the method according to the invention on the incorporation of the active agent into the matrix.

The method herein described is used in the various cases tested by the applicant who will have adapted the temperatures to the nature of the polymers and active agents used.

### Example 2

### Comparative Tests of the Method According to the Invention with a Conventional Incorporation Method for Several Active Agent-Matrix Pairs

Several tests were carried out in order to compare the incorporation rate of the active agent into the matrix. The incorporation rate was assessed by weighing, and corresponds to the ratio between the mass of active agent added and the sum of the total masses (matrix and active agent). It is determined from the incorporated matrix.

Several active agent-matrix pairs were studied. The tests by the applicant were compared with a conventional incorporation method. This conventional incorporation method comprises only the incorporation step and does not comprise an impregnation step. In other words, the conventional incorporation tests only, but entirely, reproduce the conditions of the incorporation step of the method according to the invention.

It goes without saying that these conditions may vary according to the nature of the active agent-matrix pair and that the applicant has been able to adapt them.

The tests were carried out for different polymers, with a mixture of active agent A or B which are mixtures of active agents with different compositions.

As a reminder, active agent A comprises linseed oil, plant pyrethrum and lavandin essential oil. Active agent B comprises sweet almond oil, peppermint essential oil and cedar essential oil.

The tests follow the same protocol as Example 1, with only the incorporat ion parameters changing based on the nature of the active agent-matrix pair.

### Test 1:

Active agent/matrix pair: Active agent A, EVA.

Impregnation parameters: CO2, 25°C, 30 bar, 120 min.

Incorporation parameters: 61 °C, 2 h 30 min.

### Test 2:

Active agent/matrix pair: Active agent B, Polyamide.

Impregnation parameters: CO2, 25 °C, 30 bar, 120 min.

Incorporation parameters: 70 °C, 3 h.

### Test 3:

Active agent/matrix pair: Active agent A, Polyethylene.

Impregnation parameters: CO2, 25 °C, 30 bar, 120 min.

Incorporation parameters: 90 °C, 3 h.

### Test 4:

Active agent/matrix pair: Active agent E, Casein-based biodegradable polymer.

Impregnation parameters: CO2, 25 °C, 30 bar, 120 min.

Incorporation parameters: 30 °C, 5 h.

### Test 5:

Active agent/matrix pair: Active agent A, PEBA.

Impregnation parameters: CO2, 25 °C, 30 bar, 120 min.

Incorporation parameters: 61 °C, 3 h 15 min.

### Test 6:

Active agent/matrix pair: Active agent A, Polypropylene.

Impregnation parameters: CO2, 25 °C, 30 bar, 120 min.

Incorporation parameters: 90 °C, 3 h.

### Test 7:

Active agent/matrix pair: Active agent A, Polyester.

Impregnation parameters: CO2, 25 °C, 30 bar, 120 min.

Incorporation parameters: 70 °C, 2 h 30 min.

### Test 8:

Active agent/matrix pair: Active agent A, Polyethylene.

Impregnation parameters: CO2, 25 °C, 30 bar, 120 min.

Incorporation parameters: 61 °C, 3 h.

### Results:

For each of these tests, the applicant was able to demonstrate that the use of a gas in the non-supercritical state to impregnate the matrix prior to incorporating active agent, resulted in a consistently higher rate of incorporated active agents in the matrix resulting from the method according to the invention with respect to a matrix resulting from the method carried out without the prior CO2 impregnation step, irrespective of the active agent-matrix pair selected.

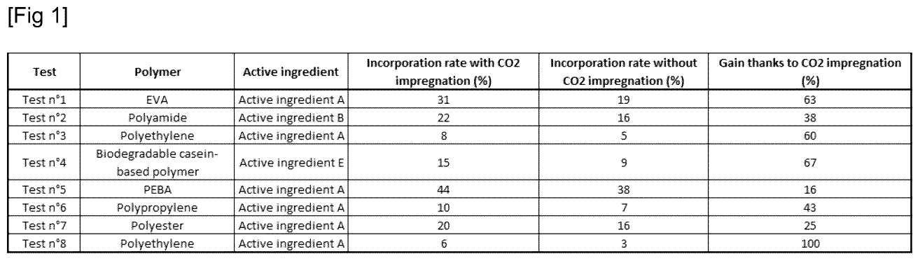

All the results are presented in the table in FIG. 1.

All of the results show a percentage increase in the incorporation rate of active agents of at least 15% and up to 100% depending on the nature of the polymers.

### Example 3

### Analysis of Impregnation Pressure on the Maximum Incorporable

Several tests were carried out in order to be able to measure the maximum active agent incorporable into the impregnated polymer matrix, based on the impregnation pressure.

The impregnation method is that described in Example 1, carried out with a gas pressure applied to the granules varying from atmospheric pressure (1 bar), i.e. no pressure applied, to a pressure of 35 bar during the impregnation phase.

The incorporation method applied is that of Example 1.

The tests were carried out using EVA and the mixture of active agent A as polymer, according to the parameters described in Example 2, Test 1.

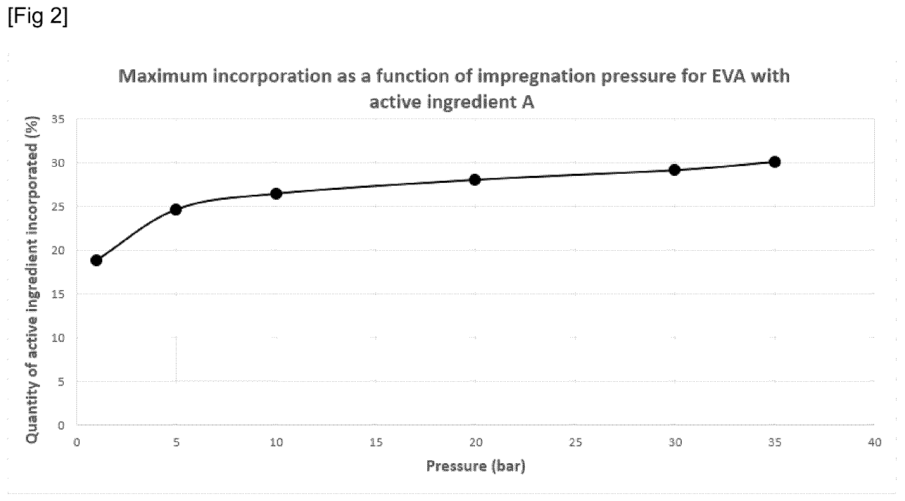

FIG. 2 shows that from a gas impregnation pressure of 5 bar, the maximum incorporable already reaches a higher value than that obtained in the conventional method without an impregnation step (see table in FIG. 1). The applicant was able to demonstrate that when performing gas impregnation over a pressure range of between 5 and 35 bar, the maximum polymer incorporation increased based on the pressure. The applicant has determined that above 35 bar, the effect on incorporation seems to reach a plateau and that between 1 bar (atmospheric pressure) and 5 bar, changes in incorporation are not significant. The pressure selected in the other examples of the method according to the invention is preferentially set at 30 bar.

### Example 4

### Comparative Tests of the Method According to the Invention with a Conventional Incorporation Method with Difficult-to-Incorporate Active Agents

In these tests, the active agents are selected from neem oil, icaridin or sweet almond oil. The conduct of the method according to Example 1 is followed for incorporation into an EVA-based matrix. These active agents are incorporated on their own, not in a mixture and are described as being difficult to incorporate as such in a polymer.

The temperature, pressure and time parameters used are as follows:
Active agent/polymer pair: Neem oil/EVA

Impregnation parameters: CO2, 25 °C, 30 bar, 120 min.

Incorporation parameters: 63 °C, 6 h.
Active agent/polymer pair: Icaridin/EVA:

Impregnation parameters: CO2, 25 °C, 30 bar, 120 min.

Incorporation parameters: 63 ° C, 6 h.
Active agent/polymer pair: Sweet almond oil/EVA

Impregnation parameters: CO2, 25 °C, 30 bar, 120 min.

Incorporation parameters: 63 °C, 6 h.

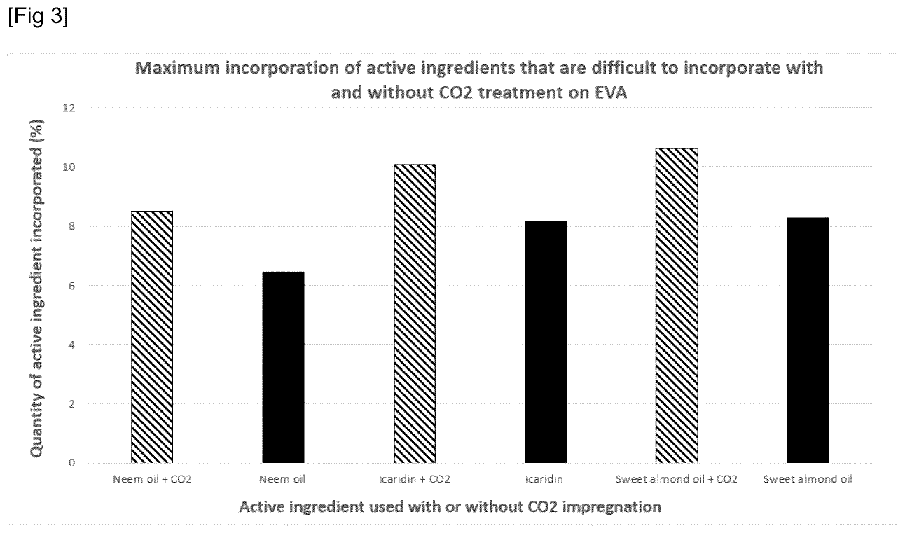

The comparative results FIG. 3 show that the method according to the invention enables much better incorporation of these active agents with respect to a conventional incorporation method. The impregnation step of the method of the applicant therefore offers a significant gain in the incorporation of these difficult active agents.

Indeed, for each of these difficult-to-incorporate active agents, the amount of active agent incorporated using the method according to the invention is increased by at least 20% compared to the amount of active agent incorporated using a method without a CO2 impregnation step.

### Example 5

### Maintaining the Matrix Incorporation Rate Over Time After Impregnation According to the Method According to the Invention

In order to determine whether the effect of the impregnation step was transient, the applicant was able to assess the durability of the matrix impregnation over time.

To this end, tests to assess maximum incorporation according to Examples 1 and 2 were carried out on a batch of EVA granules previously impregnated according to the method of the invention. The same active agents A and B were tested, but also an active agent C comprising lavender, geraniol and pyrethrum.

The protocol used is as follows:

After having selected the polymer matrix, the impregnation step is performed with carbon dioxide (CO2) or nitrogen (N2) at a temperature of 25 °C and at a pressure of 30 bar.

Once the matrix has been impregnated, a fraction of said matrix is taken to perform an incorporation test noted t0. The remaining impregnated material is stored in an open container, allowing total desorption of the selected gas.

Once incorporation has taken place at to, the amount of active agent incorporated is measured. This test is repeated once a week over a period of 4 to 5 weeks, each time taking a new matrix fraction from the open storage container.

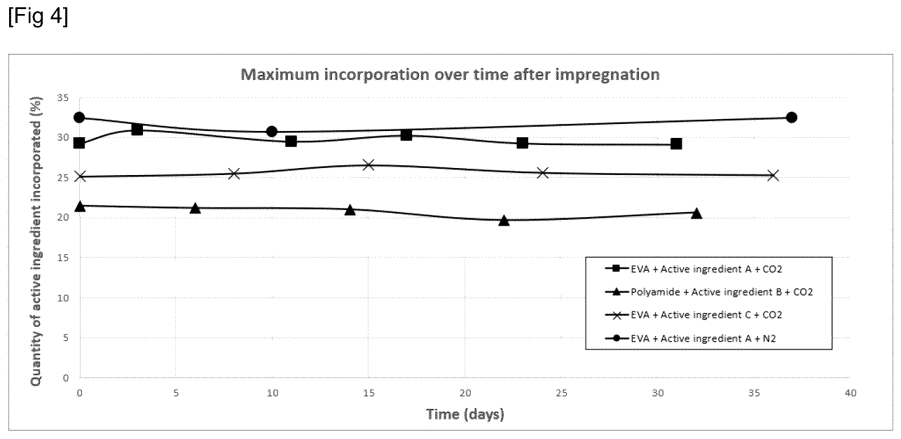

FIG. 4 exemplifies these results.

The applicant has thus been able to demonstrate that once the matrix has been impregnated, the incorporation abilities thereof remain unchanged and are still improved with respect to a conventional method. Indeed, linearity can be observed on the various incorporation tests for different active agent-matrix pairs. Impregnated matrices can thus be stored and preserved at the end of this first impregnation step, without incorporated active agent, and will retain improved incorporability over time until at least 30 days after the impregnation step.

### Example 6

### Study of the Influence of the CO₂ Impregnation Step Based on the Incorporation Temperature

In order to study the influence of the impregnation step of the CO2 in the non-supercritical state on the incorporation temperature, the applicant has carried out several comparative tests at different temperatures for incorporation of active agents into the matrix, in which an evaluation of the maximum incorporation according to the method of the invention was performed.

### These Tests Were Carried out With the Following Active Agent-matrix Pair: Active Agent A-EVA.

The maximum amount of active agent incorporated was evaluated based on the temperature and 4 tests were carried out at temperatures of 35 °C, 40 °C, 50 °C and 63 °C respectively. These 4 tests were carried out on a matrix impregnated with CO₂ and on an unimpregnated matrix in order to study the influence of CO₂ on active agent incorporation.

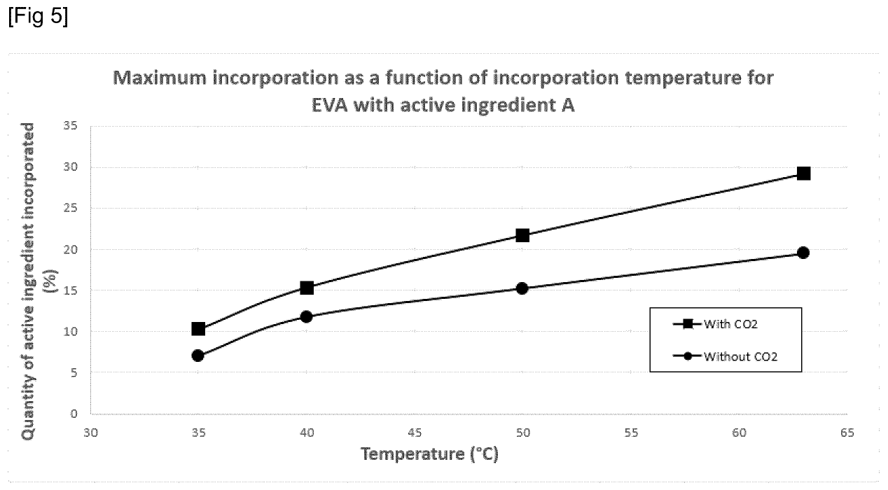

FIG. 5 shows that impregnating the matrix with CO₂ improves the amount of active agent incorporated, whatever the incorporation temperature.

As a result, a further advantage of the method according to the invention is demonstrated: a given and defined amount of active agent can be incorporated at a lower temperature, if required, if the method according to the invention is used. The curves show that actually with a method without CO2 impregnation, the maximum amount of active agent incorporated of 15% (by weight of active agent with respect to the total weight of the incorporated matrix) active agent A is reached at an incorporation temperature of 50 °C. With the method according to the invention, this value of 15% is reached at an incorporation temperature of 40 °C, i.e. much lower. For the same amount of active agent incorporated, the method according to the invention enables a lower incorporation temperature to be used with respect to the conventional method which is a significant advantage. Indeed, the method therefore makes it possible to incorporate numerous active agents that are heat-sensitive and/or volatile, without the risk of degradation or evaporation. Furthermore, heating to a lower temperature saves time in the performance of the overall method. This is an industrial advantage.

### Example 7

### Study of the Method According to the Invention on the Maximum Incorporation of Different Active Agents on a Biodegradable Polymer

The applicant sought to demonstrate that the method enabled better incorporation of active agents on different types of polymers, including biodegradable polymers,

The impregnation conditions are those of Example 2.

The incorporation parameters for all the active agents tested are: temperature 70 °C, incorporation time 6 h.

The biopolymer used is a biopolyester and the active agents tested are active agents A, C and D as described previously.

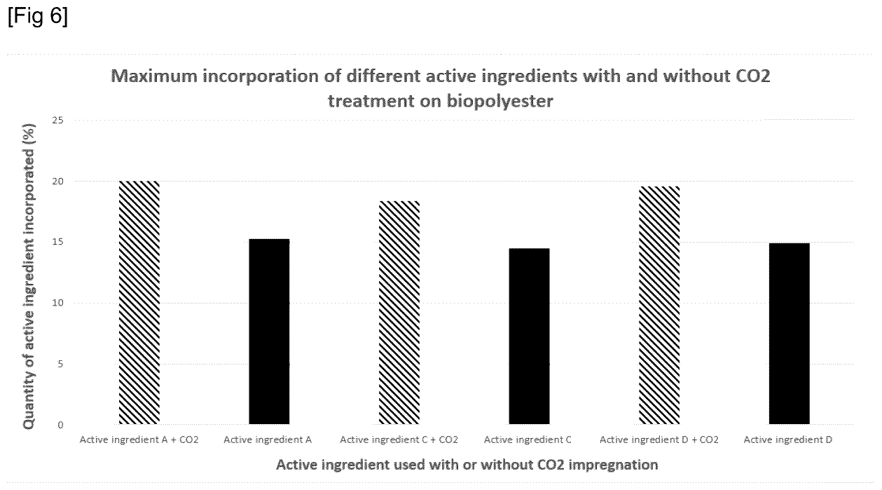

The results in FIG. 6 show that the method according to the invention once again enables an improvement in the amount of active agent incorporated with a biopolyester-type polymer for the three types of mixture of active agents tested.

### Example 8

### Study of the Method According to the Invention with Active Agents of the Chemical Pharmaceutical Type on Different Polymer Matrices

The applicant sought to demonstrate the efficacy of the method with different types of chemical pharmaceutical active agents on different polymer matrices.

The polymers used are non-degradable, namely EVA and a polyamide, and biodegradable, with a biopolyester.

The active agents tested are various pyrethroid-type insecticides or repellents or organophosphorus molecules.

The impregnation conditions are those of Example 1. The conditions for incorporating active agents are as follows:
Active agent/polymer pair: Permethrin/EVA

Incorporation parameters: 63 °C, 5 h.
Active agent/polymer pair: Phenothrin/EVA

Incorporation parameters: 63 °C, 5 h.
Active agent/polymer pair: Diazinon/EVA

Incorporation parameters: 63 °C, 5 h.
Active agent/polymer pair: Diazinon/Biopolyester

Incorporation parameters: 70 °C, 5 h.
Active agent/polymer pair: Diazinon/Polyamide

Incorporation parameters: 70 °C, 5 h.

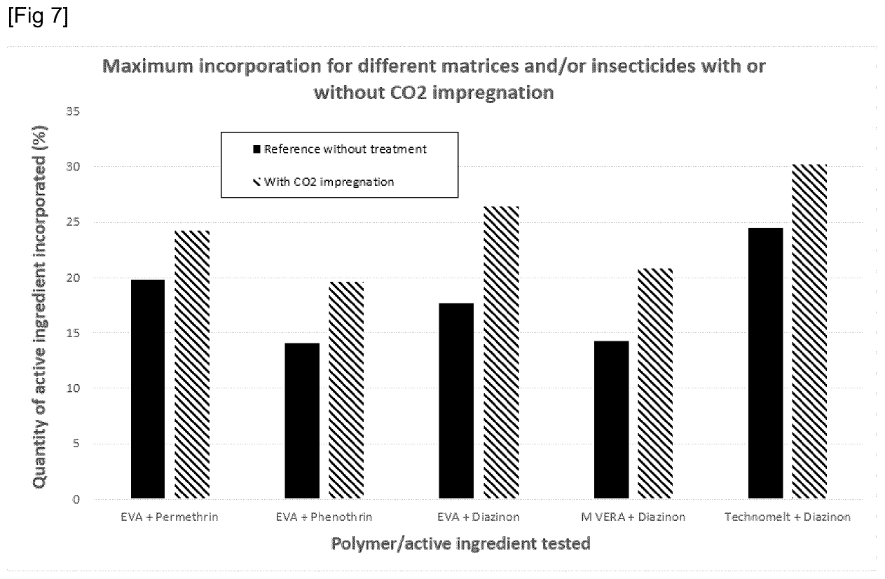

The results in FIG. 7 confirm that the method according to the invention enables an increase in the amount of active agent incorporated, whatever the nature of the active agents or the polymer matrix.

### Example 9

### Cumulative Amount of Active Agent a Released Over Time

The polymer matrices obtained according to the method of the invention are used for the treatment of a subject in need, by bringing said incorporated impregnated polymer matrix into contact with said subject so that it releases and diffuses the incorporated active agents over time.

It is therefore important to check that the matrix obtained is capable of releasing the incorporated active agents.

Measurements of the cumulative amount of active agent released by the matrix obtained by the method according to the invention were carried out over time between day 1 (D1) and day 180 (D180), and are compared with those obtained with an incorporated polymer matrix, produced by direct incorporation of active agent without an impregnation step.

The polymer used herein is EVA and the incorporated active agent is active agent A as defined previously.

The matrix produced by direct incorporation without an impregnation step incorporated a maximum of 19% active agent A (as described in the table in FIG. 1).

With the method according to the invention, the matrix obtained advantageously incorporated up to 31% of active agent A within the EVA matrix (as described in the table in FIG. 1).

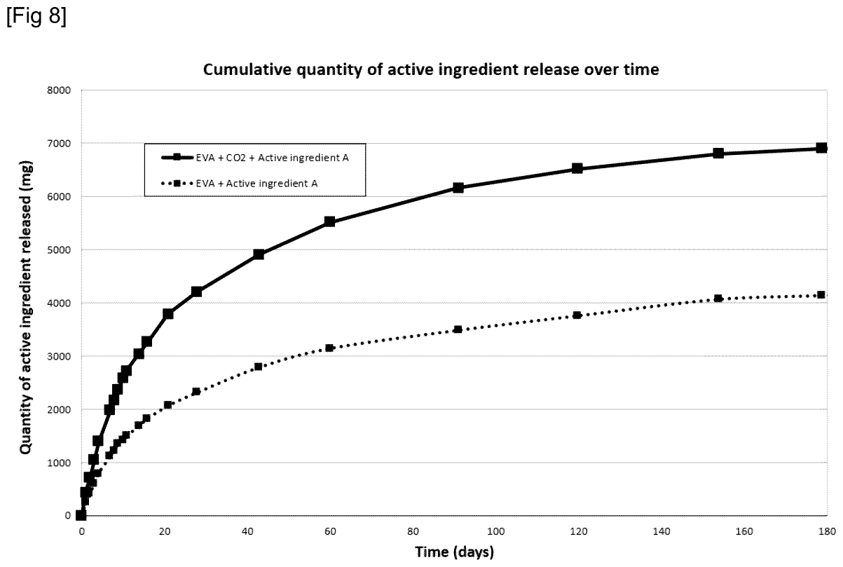

FIG. 8 shows the cumulative amount of active agent released based on time, by the two matrices produced. It is observed that:
on the one hand, the matrix obtained by the method according to the invention releases a greater amount as early as D1 than the matrix obtained by the conventional method without a prior gas impregnation step.
On the other hand, the cumulative amount of active agent released remains higher over a longer period for the matrix obtained according to the invention. Indeed, at 20 days, it is noted that the matrix obtained by the method according to the invention released about 3700 mg of active agent compared with 2000 mg with the conventional method. At D153, the matrix obtained by the method according to the invention released 6793 mg compared with about 4000 mg with the conventional method.

The matrix obtained according to the method of the invention therefore not only enables improved incorporation of active agent but also releases said active agent in greater amounts over a period of up to several months. No sequestration of the active agent by said matrix is observed, said active agent is correctly released. It can therefore be concluded that the polymer matrix impregnated and incorporated using the method according to the invention allows long-term treatment with greater amounts of active agent released. Thus the matrix obtained according to the method of the invention makes it possible to obtain a more efficient delivery device.

### Example 10

### Cumulative Amount of Active Agent B Released Over Time

The polymer matrices obtained according to the method of the invention are used to treat a subject in need, by bringing said incorporated impregnated polymer matrix into contact with said subject so that it releases and diffuses the incorporated active agents over time.

It is therefore important to check that the matrix obtained is capable of releasing the incorporated active agents. Measurements of the cumulative amount of active agent released by the matrix obtained by the method according to the invention were carried out over time between day 1 (D1) and day 196 (D196), and are compared with those obtained with a conventional incorporated polymer matrix, produced by direct incorporation of active agent without an impregnation step. The polymer used herein is a polyamide and the incorporated active agent is active agent B as defined previously.

The matrix produced by direct incorporation without an impregnation step incorporated a maximum of 16% active agent B (as described in the table in FIG. 1).

With the method according to the invention, the matrix obtained advantageously incorporated up to 22% of active agent B within the polyamide matrix (as described in the table in FIG. 1).

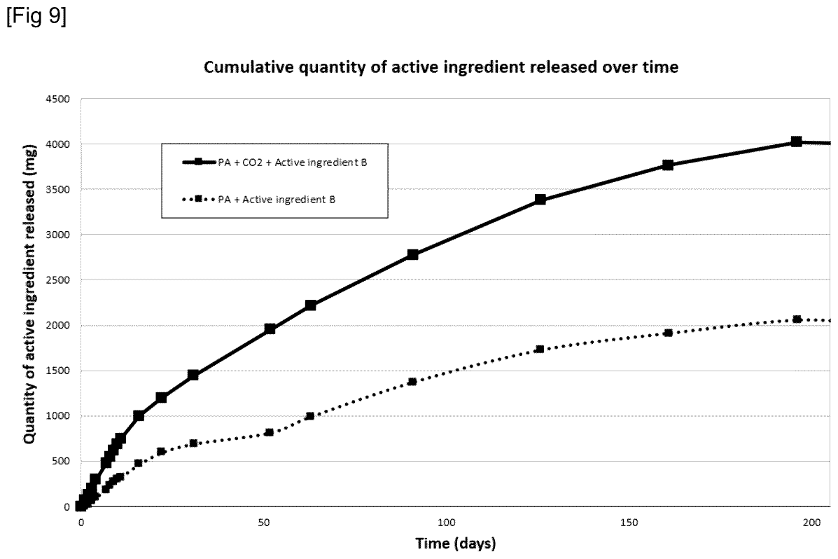

FIG. 9 shows the cumulative amount of active agent released based on time, by the two matrices produced. It is observed that:
on the one hand, the matrix obtained by the method according to the invention releases a greater amount as early as day 1 than the matrix obtained by the conventional method without a prior gas impregnation step.
On the other hand, the amount of active agent released remains higher over a longer period. Indeed, at 30 days, it is noted that the matrix obtained by the method according to the invention releases 1450 mg per day compared with about 680 mg with the conventional method.

The matrix obtained according to the method of the invention therefore not only enables improved incorporation of active agent but also releases said active agent in greater amounts over a period of up to several months. No sequestration of the active agent by the matrix is observed, said active agent is correctly released. It can therefore be concluded that the polymer matrix impregnated and incorporated using the method according to the invention leads to long-term treatment with greater amounts of active agent released. Thus the matrix obtained according to the method of the invention therefore makes it possible to obtain a more efficient delivery device.

### Example 11

### Comparison of the Method According to the Invention in 2 Successive Steps with the 1-Step Method

The applicant has tried to combine the impregnation of the matrix with a non-supercritical fluid and the incorporation of said active agents into the same step. It was thus found that the incorporation could not be carried out. Impregnation carried out at the same time as incorporation of an active agent therefore does not allow incorporation of said active agent. Surprisingly, only the method in two successive steps according to the invention offers improved active agent incorporation properties.

### Example 12

### Scanning Microscopy Analysis of the Polymer Matrix Obtained According to the Method

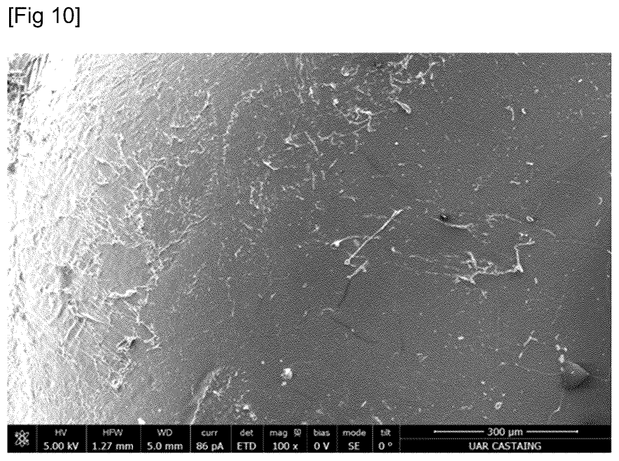

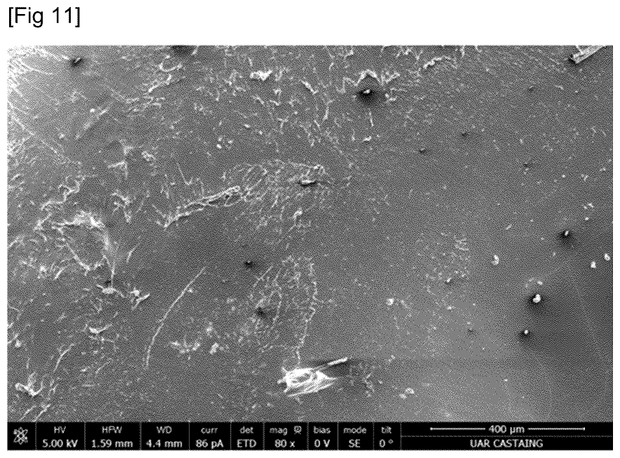

Scanning microscopy analyses were carried out on the polymer matrix non-impregnated then impregnated with the CO2 gas according to the invention. Surprisingly, the images obtained in FIGS. 10 and 11 showed no porosity of the matrix before or after CO2 treatment. Surprisingly, impregnating the matrix with the gas according to the method of the invention does not therefore confer, contrary to what is known in the state of the art, a porous structure to the matrix which could have explained an improvement in the amount of incorporated active agent.

### Example 13

### Comparative Tests of the Method According to the Invention with a Conventional Incorporation Method Using an EVA-type Polymer and an Essential Oil

The impregnation and incorporation parameters for the method according to the invention applied to this matrix are as follows:

Active agent/matrix pair: Lavandin essential oil, EVA.

Impregnation parameters: CO2, 25 °C, 30 bar, 120 min.

Incorporation parameters: 63 °C, 5 h.

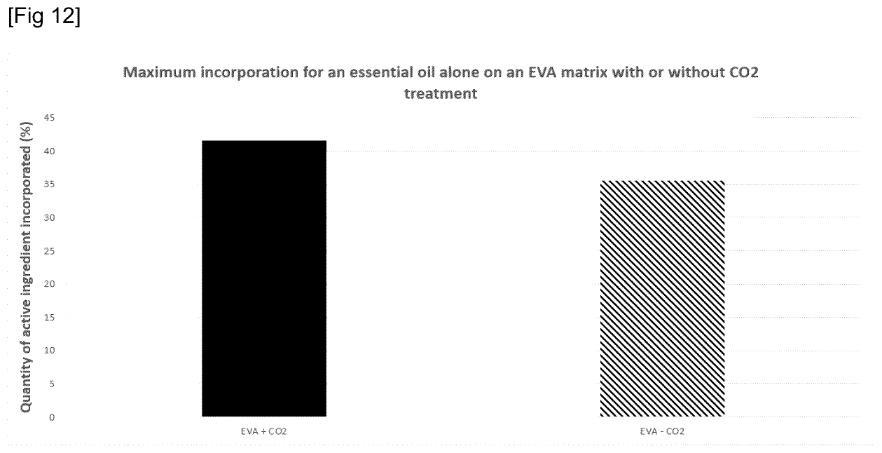

The results in FIG. 12 confirm that the method according to the invention increases the incorporated amount of an essential oil only within a polymer matrix, compared with that obtained by means of a conventional incorporation method without a CO2 impregnation step.

1. A method for producing a thermoplastic polymer matrix loaded with at least one lipophilic liquid active agent by means of a gas characterized in that it comprises the following successive steps:
a) Impregnating the polymer with said gas in the non-supercritical state selected from carbon dioxide (CO2) or dinitrogen (N2) at a temperature strictly less than 40 °C and at a pressure of between 5 and 35 bar;
b) Incorporating the active agent at a temperature of between 30 °C and 100 °C

2. The method according to claim 1, wherein the gas impregnation step is carried out at a temperature of between 15 °C and 35 °C.

3. The method according to claim 2, wherein the gas impregnation step is carried out at a temperature between 20 °C and 30 °C.

4. The method according to claim 2, wherein the duration of the impregnation step is between 5 min and 150 min.

5. The method according to claim 1 wherein the polymer is a thermoplastic selected from ethylene vinyl acetate (EVA), polyamides, polyether block amides (PEBA), polypropylenes (PP), polyethylenes (PE), thermoplastic polyurethanes (TPU), polyvinyl chlorides (PVC), polystyrenes such as polystyrene-block-poly(ethylene-ran-butylene)-block-polystyrene (SEBS) or polystyrene-block-polyisoprene-block-polystyrene (SIS), polyesters, polylactic acids (PLA) or else biodegradable polymers, agro-polymers, bioplastics or biosourced thermoplastics, biopolyesters.

6. The method according to claim 1, wherein the incorporated active agent is selected from chemical pharmaceutical active agents, active agents of natural origin, essential oils, vegetable oils or mixtures thereof.

7. Use of a gas in the non-supercritical state selected from carbon dioxide (CO2) or dinitrogen (N2) to impregnate a thermoplastic polymer matrix, characterized in that said gas is impregnated at a temperature strictly less than 40 °C and at a pressure of between 5 and 35 bar to increase the amount of lipophilic liquid active agent incorporated into said thermoplastic polymer matrix.
